(19) United States
(12) Patent Application Publication
(10) Pub. No.: US 2025/0104878 A1
(43) Pub. Date: Mar. 27, 2025

(54) DATA PROCESSING APPARATUS AND METHOD

(71) Applicant: CANON MEDICAL SYSTEMS CORPORATION, Tochigi (JP)

(72) Inventors: Joseph S. BOYLE, Edinburgh (GB); Alison O'NEIL, Edinburgh (GB)

(73) Assignee: CANON MEDICAL SYSTEMS CORPORATION, Tochigi (JP)

(21) Appl. No.: 18/891,490

(22) Filed: Sep. 20, 2024

Related U.S. Application Data
(60) Provisional application No. 63/585,765, filed on Sep. 27, 2023.

(51) Int. Cl. G16H 70/20; G06F 40/14; G06F 40/40

(52) U.S. Cl. CPC G16H 70/20; G06F 40/14; G06F 40/40

(57) ABSTRACT

A data processing apparatus for obtaining an output corresponding to a medical text input, the apparatus comprises processing circuitry configured to:
provide a medical text input, or data derived from the medical text input, to a trained model;
provide instructions to the trained model to repeatedly assess layers of a hierarchical ontology that comprises a plurality of nodes at each of a plurality of layers, thereby to determine path(s) through the layers via node(s) in successive layers that are connected according to the hierarchical ontology and that match the medical text input;
select at least one node from the node(s) at the end or other point(s) of the determined path(s), or select no nodes if there are no suitable matches of the medical text input to nodes; and
output text, or other data, associated with the selected at least one node.

### FIELD

Embodiments described herein relate generally to a method and apparatus for processing data, for example for assigning codes to a patient's medical record or performing other navigation or processing of a hierarchical ontology in an automated fashion.

### BACKGROUND

Clinical coding is the task of assigning a set of medical codes to a patient's health record after an incident of care, for instance a stay at the intensive care unit ICU. These codes are used primarily for reimbursement of health institutions but may also be used for billing, audit, resource management, epidemiological study, measurement of treatment effectiveness, and other purposes. The task of assigning diagnostic International Classification of Disease (ICD) codes to patient hospital admissions is typically performed by expert human coders. ICD diagnostic codes may, for example, be assigned to a patient's discharge note. Automatic methods predominantly use supervised deep learning. Supervised methods struggle to learn rare codes for which there are few or no training examples, and struggle to generalize to new data.

Coding is usually a manual process, performed by specialists via inspection of a patient's medical documentation. Codes are usually manually assigned by specialists known as ‘diagnostic coders’ who read patient documentation including discharge letters and assign relevant codes. This can be a time-consuming and error-prone process; studies report the costs related to medical coding to be billions of dollars per year in the US alone. Thus, automation of coding has been pursued since the 1990s, with deep learning techniques currently dominating. The assignment frequency of ICD codes follows a long-tailed distribution. The ICD-10-CM ontology contains 96,000 distinct codes, of which 73,000 are assignable. There are thousands of codes with few or no training examples. Further, ICD codes often have considerable conceptual overlap with their peers, making it difficult to learn distinct representations without large volumes of labelled training data. Traditional supervised deep learning techniques struggle with ICD codes seen rarely or not seen in their training dataset. Generative large language models like GPT-3 have been used for clinical tasks, such as question answering, summarization, and clinical information retrieval without the need for any task specific training. Getting a GPT model to perform tasks generally involves crafting an input text prompt to describe the task and subsequently interpreting or checking its response.

Since supervised learning techniques struggle with rare ICD codes, there is potential for using off-the-shelf pre-trained generative LLMs with no task-specific training for the purpose of zero-shot and few-shot code assignment. However, using a naive prompt such as one that states “you are a clinical coder . . . ” before assigning a clinical coding task results in poor outcomes. In particular, the model frequently responds with an incorrect ICD code for the description that it provides in response.

### BRIEF DESCRIPTION OF THE DRAWINGS

Embodiments are now described, by way of non-limiting example, and are illustrated in the following figures, in which:

FIG. 1 is a schematic illustration of an apparatus in accordance with an embodiment;

FIG. 2 is a schematic of a hierarchical ontology in accordance with an embodiment;

FIG. 3 is an image showing the input to and output of a method of processing data according to an embodiment;

FIG. 4 is an image showing the input to and output of a method of processing data according to an embodiment; and

FIG. 5 shows the tabulated results of a performance comparison between the method of text processing according to an embodiment and some state of the art methods of processing text.

### DETAILED DESCRIPTION

According to certain embodiments there is provided a data processing apparatus for obtaining an output corresponding to a medical text input, the apparatus comprising processing circuitry configured to:
provide a medical text input, or data derived from the medical text input, to a trained model;
provide instructions to the trained model to repeatedly assess layers of a hierarchical ontology that comprises a plurality of nodes at each of a plurality of layers, thereby to determine path(s) through the layers via node(s) in successive layers that are connected according to the hierarchical ontology and that match the medical text input;
select at least one node from the node(s) at the end or other point(s) of the determined path(s), or select no nodes if there are no suitable matches of the medical text input to nodes; and
output text, or other data, associated with the selected at least one node.

According to certain embodiments there is provided a data processing method comprising:
providing a medical text input, or data derived from the medical text input, to a trained model;
providing instructions to the trained model to repeatedly assess layers of a hierarchical ontology that comprises a plurality of nodes at each of a plurality of layers, thereby to determine path(s) through the layers via node(s) in successive layers that are connected according to the hierarchical ontology and that match the medical text input;
selecting at least one node from the node(s) at the end or other point(s) of the determined path(s), or selecting no nodes if there are no suitable matches of the medical text input to nodes; and
outputting text, or other data, associated with the selected at least one node.

In various embodiments described herein, the approach of using off-the-shelf generative large language models (LLM) which have been trained using self-supervised learning on upto trillions of tokens is used. A language model (LM) is a machine learning model, such as a Neural Network (NN) for example, that is usually trained primarily on text data. LLMs may have billions of parameters and be trained using very large amounts of text. A generative LLM, or autoregressive language model, takes a sequence of text data as input and returns a new sequence as output, generated using the current input plus the previous parts of its response. OpenAI's GPT models are currently the state-of-the-art in generative LLMs. The latest multimodal GPT-4 model has been extended beyond text inputs to include image inputs. Zero- and few-shot prediction refers to the ability to predict labels which were not seen during training or for which only a few training samples were seen.

Large language models (LLMs) are used in some embodiments to develop a practical solution for ICD coding that is suitable for zero-shot and few-shot code assignment.

Generative LLMs are used to perform the task of ICD coding even in the absence of training examples i.e. with no task-specific training. LLMs are used to perform ICD coding by guiding a search for clinical entities through an ICD diagnostic tree. This can be conceptualized as using an LLM as the discriminator function in a multi-label decision tree. Prompts are used to communicate with and direct the behaviour of Large Language Models (LLMs). A prompt may comprise an input text sequence created by a human user designed to elicit a desired response from the LLM for a target task. They serve as inputs or queries that users can provide to elicit specific responses from a model. In some embodiments, these prompts may be in natural languages. The act of prompting an LLM may be seen as giving instructions to the LLM. In this framework, the LLM is prompted to pass judgement as to the relevance of each branch of the ICD tree based on its text description.

Methods which leverage the power of generative LLMs but explicitly insert knowledge of the ICD ontology in the prompt and search strategy may potentially be used to handle the challenge of zero-shot prediction, generalise across ICD revisions and predict without training on restricted patient data.

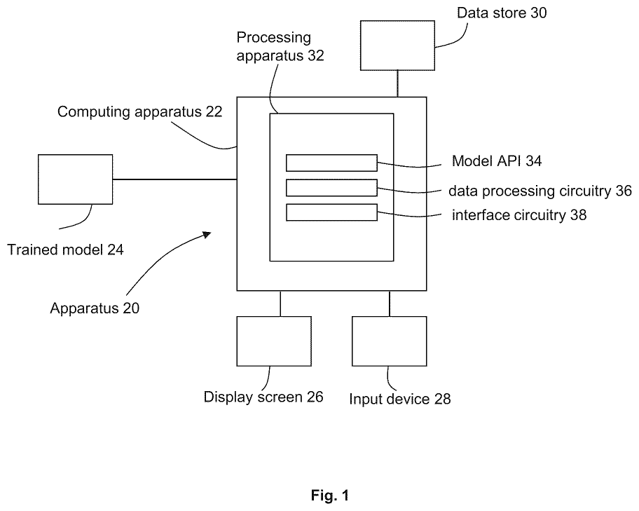

A data processing apparatus 20 according to an embodiment is illustrated schematically in FIG. 1. In the present embodiment, the data processing apparatus 20 is configured to process medical text data for obtaining an output corresponding to a medical text input. In other embodiments, the data processing apparatus 20 may be configured to process any other appropriate data.

The data processing apparatus 20 comprises a computing apparatus 22, which in this case is a personal computer (PC) or workstation. The computing apparatus 22 is connected to a display screen 26 or other display device, and an input device or devices 28, such as a computer keyboard and mouse.

The computing apparatus 22 is configured to obtain data from a data store 30. The data have been obtained or generated using any suitable apparatus or from any suitable source. The data in the embodiments comprises medical text. The medical text may comprise at least one of medical notes for a patient or other subject, results of a diagnostic or other procedure, test or scan results or text associated with such results. The medical text may comprise a discharge note for the patient, detailing the medical treatment received by the patient or other subject during a visit to a medical facility. The medical text may further comprise an in-context training example that modulates the format of the output and/or instructs the LLM to provide a particular output.

The computing apparatus 22 may receive data from one or more further data stores (not shown) instead of or in addition to data store 30. For example, the computing apparatus 22 may receive data from one or more remote data stores (not shown) which may form part of a Picture Archiving and Communication System (PACS) or other information system.

Computing apparatus 22 provides a processing resource for automatically or semi-automatically processing the data. Computing apparatus 22 comprises a processing apparatus 32. The processing apparatus 32 comprises an Application Programming Interface (API) 34 that provides an interface for communication with a trained model 24. The apparatus also includes data processing circuitry 36 configured to perform processes including sending instructions to the trained model 24 and receiving outputs from the trained model 24, via the API 34. The apparatus also includes interface circuitry 38 configured to obtain user or other inputs and/or to output results of the data processing.

The API 34 may be stored permanently at the computing apparatus 22 or data store 30, or may be downloaded from a remote source and executed in dynamic fashion during a processing session performed under control of the data processing circuitry 36. Interface circuitry 38 processes data input and output, and in particular, controls data flow to and from the display screen 26 and input device 28.

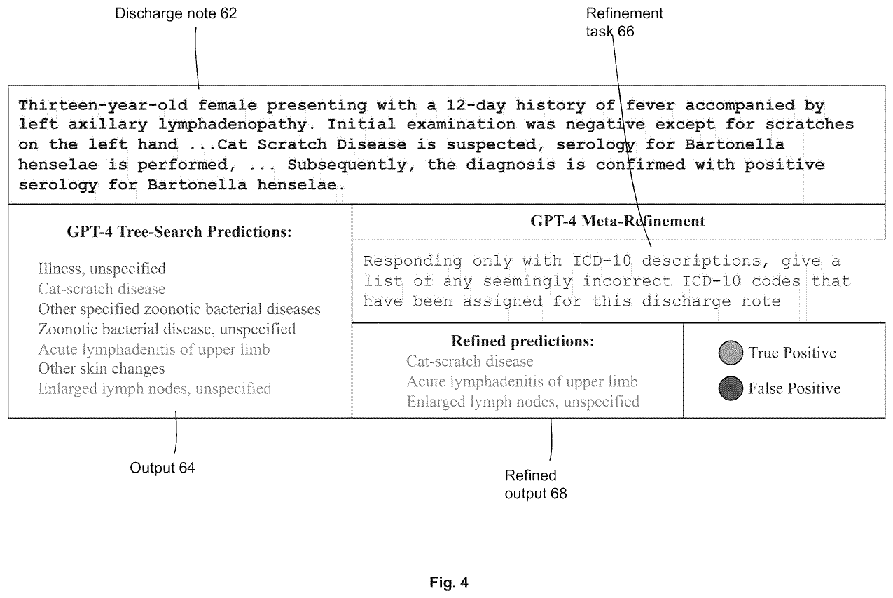

The trained model 24 in the embodiment of FIG. 4 is a large language model (LLM), in this case GPT-4, located on a server remote from the apparatus 22. Communication between the computing apparatus 22 and the trained model may be via the internet or any other suitable communication or networking system. In other embodiments, the trained model may be stored or implemented locally at the apparatus 20.

In other embodiments, any suitable trained model may be used, for example any suitable language model or large language model. In some embodiments the trained model comprises any of GPT-2, GPT-3.5, GPT-4, PaLM, LLaMa, BLOOM, Ernie, T5, Claude or Claude 2 or any suitable derivatives or developments thereof.

In some embodiments, the trained model is in the form of a set of trained models each of which may be used separately or in combination.

In the present embodiment, the circuitries 34, 36, 38 are each implemented in computing apparatus 22 by means of a computer program having computer-readable instructions that are executable to perform the method of the embodiment. However, in other embodiments, the various circuitries may be implemented as one or more ASICs (application specific integrated circuits) or FPGAs (field programmable gate arrays).

The computing apparatus 22 also includes a hard drive and other components of a PC including RAM, ROM, a data bus, an operating system including various device drivers, and hardware devices including a graphics card. Such components are not shown in FIG. 1 for clarity.

The data processing apparatus 20 of FIG. 1 is configured to perform methods as illustrated and/or described in the following.

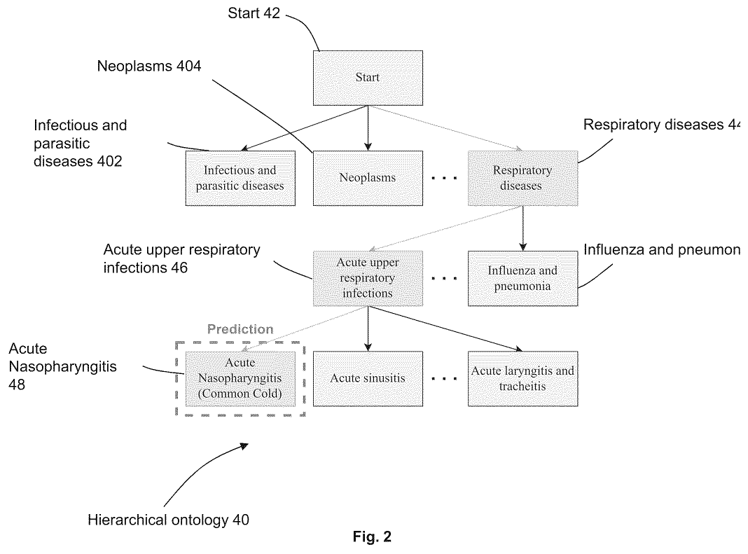

FIG. 2 shows a schematic of a hierarchical ontology 40 or tree data structure that comprises multiple hierarchical layers, each layer comprising multiple nodes. In this embodiment, the ontology comprises a subset of the ICD-10 diagnostic tree. The ontology shown has three layers and each layer comprises two to three nodes. Other embodiments may comprise more or fewer layers and/or nodes. The ICD diagnostic tree contains nodes, wherein each node comprises at least one code (not shown), and at least one description of the code. Code descriptions for each node are shown in FIG. 2. The codes in the ICD-10 diagnostic tree are alphanumeric while the code descriptions comprise natural language. The code may be a procedure code or a diagnostic code. In the current embodiment, the codes are diagnostic codes. These codes are associated with each node in the tree data structure as shown in FIG. 2 and the code descriptions within a tree branch may be highly correlated. Each successive layer may comprise a code and code description that corresponds to a successively specific medical diagnosis or procedure. The code descriptions ‘Respiratory diseases 44’, ‘Acute upper respiratory infections 46’ and ‘Acute Nasopharyngitis 48’ in particular are labelled in FIG. 2 as part of a method for processing text data that will be discussed in more detail subsequently. Some nodes may be assignable to an input medical text and some may not. A node that has no further branches below it may be referred to as a leaf node. Typically, a leaf node is assignable. The hierarchical ontology may comprise one or more of the International Classification of Disease (ICD), SNOMED CT, Radlex or other diagnostic code ontology.

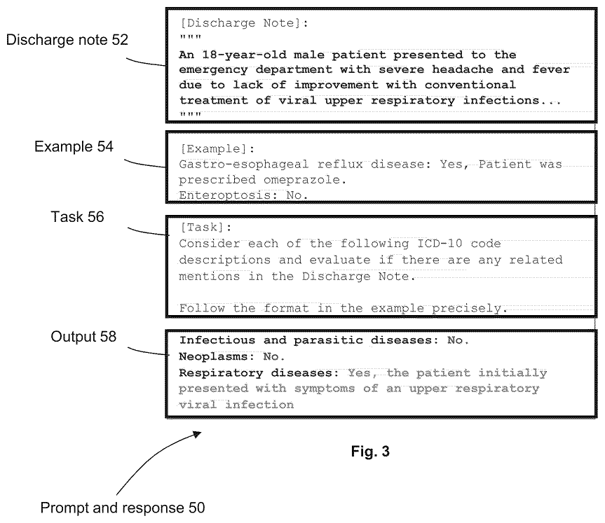

FIG. 3 shows a prompt and response 50 according to an embodiment of the data processing apparatus 20. The prompt and response 50 comprises the medical text input provided to, and the output 58 received from, the data processing apparatus 20. The medical text input provided to the apparatus comprises the text data and instructions (in the form of text) provided to the trained model 24. The set of inputs provided to the apparatus 20 is also referred to as the prompt. The prompt may include medical notes for a patient or other subject, a result of a diagnostic step or a medical procedure, results of test scans and other text associated with clinical procedures. In this embodiment, the input to the apparatus 20 comprises a discharge note 52, an example 54 and a task 56. In other embodiments, the input to the apparatus 20 may comprise any text, including medical text or any data derived from the medical text. The apparatus 20 uses a trained model 24 to process the data contained in the discharge note 52, example 54 and task 56. In this embodiment, the trained model 24 is a large language model (LLM). In this embodiment, the output of the apparatus 20 is the output 58 which comprises text. The apparatus 20 uses the trained model 24 and the inputs 52, 54 and 56 to obtain the output 58. The output 58 may comprise text and/or code(s) for example for use in at least one of billing, audit, resource management, epidemiological study, measurement of treatment effectiveness, insurance processing, or enhancement of medical records.

In this embodiment, the discharge note 52 comprises information relating to the symptoms experienced by, medical treatment given to, and/or particulars of, a patient or other subject. In other embodiments, the discharge note 52 may comprise any text, including medical text or any data derived from the text or medical text. The discharge note contains text that the trained model 24 matches to the nodes in the hierarchical ontology. In particular, the text comprising the code description associated with a node may be matched with the text in the discharge note. In some embodiments, the text comprising the code description associated with a node may be matched with some or all of the text contained in the medical text input.

The example 54 in this embodiment comprises text descriptions of sample ICD codes, binary (yes/no) decisions that denote the existence or lack thereof, of particular clinical diagnoses that correspond to the sample ICD code descriptions and a text quote that supports the positive (yes) result or results of the assessment. In other embodiments, the example 54 may comprise other text. Providing the text descriptions of sample ICD code descriptions in the prompt when instructing the trained model to perform clinical coding may improve the performance of the method in contrast with prompts that do not include the example 54.

The hierarchical ontology along with the ICD codes and ICD code descriptions are available to the apparatus 20 and processed using the trained model 24 for their relevance to the text that the discharge note 52 comprises in particular, and to the contents of the prompt in general. The hierarchical ontology along with the ICD codes, descriptions, rules and other properties of the ontology may be stored in the hard drive of the apparatus 20 and provided to the trained model 24. In other embodiments, the sample ICD code descriptions may be processed for relevance to any subset or all of the data contained in the prompt. The trained model 24 used to process text data may be an LLM. The sample ICD code descriptions, binary decisions and supporting text quotes may be understood to be an example that provides context to the LLM for processing the remainder of the prompt data. They enable the trained model 24 to follow the format of the example 54 when responding to the prompt with output 58. In other words, they can for example guide the output 58 to present data in the way that it is presented in the example 54.

The task 56 in this embodiment comprises an information retrieval request. The task 56 instructs the trained model 24 to assess the relevance of the example 54 to the discharge note 52. Relevance between sets of data may be assessed on the basis of exact string matching between sets of textual data, such as the prompt and the code descriptions in the hierarchical ontology. In other embodiments, other notions of similarity or the relationship between text strings may be used to assess relevance. In some embodiments, a node may be selected as relevant if there is a match between at least some of the text associated with the node, such as the code description, and at least some of the text comprising the prompt or medical text input. Example 54 is a demonstration of the expected output format that the model should respond using, it should not necessarily be inferred that these diagnoses are present based on this information. Natural language descriptions can be quite varied for the same ICD code. The model will consider semantic similarity other than identical text (e.g. ‘related mentions’ in the task description 56). To extract binary predictions, for example yes/no, exact string matching may be used in at least some embodiments.

The output 58 comprises binary (yes/no) results, ICD code descriptions and text quotes that supports the positive (yes) result of the assessment performed by the trained model 24 on the basis of the discharge note 52 and the example 54. The output 58 follows the format of the example 54.

An embodiment is now described, with further reference to FIGS. 2 and 3, as illustrative of a single method for processing text. The method utilizes generative large language models (LLMs) as the trained model 24. The method sparsely and recursively explores the tree or hierarchical ontology 40 of ICD medical codes to find a path through the ontology and to obtain ICD code descriptions that match the contents of the prompt, or the input medical text data. Spare exploration of the tree or ontology of medical codes in order to find relevant codes improves the efficiency of the method. At each node of the ontology 40 the LLM considers if there is a match between the code description associated with the node and the contents of the prompt and uses a matching criterion to guide the path or paths taken through the ontology 40. At each node, the LLM may be prompted and its responses are used to guide the path taken through the ontology. The method comprises composing or engineering a prompt to include sample ICD code descriptions and one or more in-context training examples 54 in addition to a discharge note 52 and a task 56. The use of code descriptions and an in-context training examples increases the accuracy of code assignment. The method frames the task 56 as an information extraction/retrieval task. We do not assume that the LLM has any task-specific training or in-built knowledge of the ICD tree and we depend on the LLM's existing capabilities. During tree search, at each visited node, the descriptions of its set of child codes may be provided and it may be requested if any codes have related mentions in the target clinical document.

The method starts with providing a prompt to the apparatus 20. The prompt may comprise a medical text input or data derived from the medical text input. The prompt in FIG. 3 comprises a discharge note 52, an example 54 and a task 56 and is provided to the trained LLM. Referring to FIG. 2, the method begins at the node labelled start 42 where the prompt has already been delivered to the apparatus 20. On the basis of the task 56, the trained model 24 begins to recursively interrogate successive layers of the ontology 40 to find a match between the ICD code descriptions in the ontology and the contents of the prompt. Of the three nodes denoting ICD code descriptions shown in the first layer of the ontology 40 of FIG. 2, the trained model 24 decides that only the node Respiratory diseases 44 matches the text of the prompt. The trained model 24 hence stores a ‘yes’ response for the node Respiratory diseases 44 and a ‘no’ response for the other two nodes with ICD code descriptions ‘Infectious and parasitic diseases 402’ and ‘Neoplasms 404’. Three nodes have been shown in the first layer of the ontology 40 for illustrative purposes only. In reality, there may be more or fewer nodes in the first layer.

Since the matched node Respiratory diseases 44 is not a leaf node, it may be not assignable and so the model 24 assesses the subset of ICD code descriptions that have Respiratory diseases 44 as their parent in the next lower layer for a match with the contents of the prompt. It can be seen in the subsequent layer that the LLM chooses ‘Acute upper respiratory infections 46’ and rejects ‘Influenza and pneumonia 406’. The LLM may decide to assign a node that is a parent node rather than a leaf node if it considers that none of the leaf nodes match the prompt, as long as the parent node is assignable. In various embodiments, non-leaf codes should not be assigned, and there always exists a leaf code even if that code simply says, for example, ‘<parent-condition> unspecified’. The LLM can return ‘Yes’ for a parent-node, for example this is what happens in FIG. 2. for the two parent nodes Resp. diseases and Acute upper resp. infections. These nodes are not ‘assigned’ in the sense that they will be returned as predictions/assigned codes, since they are not leaf-codes and are non-assignable. The non-leaf codes are programmatically after the tree-search completes.

Since the Acute upper respiratory infections 46 node is not a leaf node, the method continues to assess the ICD code descriptions in the third layer of the ontology 40 which have Acute upper respiratory infections 46 as a parent node for a match with the contents of the prompt. Of the three nodes shown in the third layer of the ontology 40, the model 24 decides that the node Acute Nasopharyngitis 48 has a description that matches the contents of the prompt. Since Acute Nasopharyngitis 48 is a leaf node, it is assignable and the traversal of this particular branch of the ontology comes to an end with the trained model storing a ‘yes’ answer to the task 56 for the node Acute Nasopharyngitis 48. The trained model 24 then generates the output 58. It can be seen that the output 58 follows the format of the example 54. The output 58 recites the ICD code descriptions of the first layer of the ontology 40 followed by yes/no answers to the task 56. The output 58 further recites a supporting quote referencing the text in the prompt, in particular the text in the discharge note 52, to support the positive (yes) answer to the task 56 for the node Respiratory diseases 44. The output contains the code-descriptions and (Yes/No) answers for whatever children nodes are being processed—not just the first-layer. The first layer is shown in FIG. 3 as an example, the process is the same for subsequent nodes, regardless of which layer of the ontology is being searched. For instance, in the step after predicting ‘yes’ for ‘Respiratory Diseases’ in FIG. 2, the model will be prompted to consider each of the child code descriptions of ‘Respiratory Diseases’, which will then be recited back alongside the model's yes/no answer.

This embodiment describes a search through only one branch of the ontology 40. In other embodiments, multiple branches of the ontology may be searched due to multiple matches between the prompt text and the code descriptions. The multiple branches may be searched concurrently or sequentially. The associated output in such a case may contain multiple code descriptions that resulted in a ‘yes’ answer to the task and an equal number of supporting quotes associated with the code descriptions. In such an example, there may be more than one leaf nodes, and associated ICD codes, assigned in response to the prompt. The output of the method may potentially comprise data other than text in some variants.

This method may avoid the need for task-specific training, and instead may rely on the pre-trained LLM's pre-existing capabilities in processing natural language. The LLM may be not trained on the text that comprises the text of the code descriptions in the ontology. The LLM may be trained in text additional to the text associated with the ontology. The LLM may need to perform a zero-shot prediction when processing text that it has not been trained on. The LLM is used to guide a search through the diagnostic tree for relevant ICD code descriptions resulting in an output that corresponds to the medical text input. The LLM is iteratively prompted to consider code descriptions at increasing specificities. Ultimately, the LLM in certain embodiments follows only the paths in the tree for which all nodes obtain a “Yes” response to the task 56, meaning that the tree is sparsely explored. In some embodiments, only a subset of the nodes of the hierarchical ontology are subject to assessment by the trained model. This means that the method is efficient compared to a linear search over all codes. The LLM may be seen as a discriminator function acting on the text associated with nodes in the hierarchical ontology and selecting relevant nodes in order to obtain a path through successive layers of the ontology.

It is possible that no nodes are selected, and hence no ICD codes or code descriptions are assigned. One way in which this might happen is if there are no suitable matches in the hierarchical ontology for the medical text input, or if the LLM considers that there are no such matches.

FIG. 4 comprises inputs and outputs that illustrate the task of refinement of the output of a text processing method according to an embodiment, for example, for the method previously described with reference to FIG. 2 and FIG. 3. FIG. 4 comprises a discharge note 62, an output 64 based on the discharge note (and more generally the prompt that comprises the discharge note 62), a refinement task 66 and a refined output 68 based on the refinement task.

Refinement is performed by prompting the trained model 24 to select all of or a subset its previously assigned codes. The refinement may use the trained model 24 or another trained model. The other trained model may also be an LLM. The refinement may eliminate one or more selected nodes and/or the associated code descriptions from the results of the first assessment of the ontology.

FIG. 4 illustrates a discharge note 62 and an output 64 generated on the basis of the prompt comprising the discharge note 62. FIG. 4 shows a subset of the prompt that was used to generate the output 64. The output 64 in this example 64 is a stylized version of the actual model output containing the ‘Yes, . . . ’, ‘No, . . . ’ answers. The prompt and output format may be the same as the embodiment of FIG. 3 or have any other suitable format. The complete prompt may be similar to the first embodiment wherein the prompt also includes an example and a task. The prompt may also be different from the first embodiment. The output 64 is shown to comprise seven ICD code descriptions. A refinement task 66 is then provided to the trained model 24 or second trained model. The refinement task 66 contains instruction to the model to provide a list of seemingly incorrect ICD code descriptions from the output 64. A refined output 68 is generated by the trained model 24 or second trained model by removing the seemingly incorrect codes from the tree-search results of the first task given to the LLM. In the current embodiment, the number of predicted ICD code descriptions is reduced from 7 to 3. Codes that the model considers incorrect are removed from the set of predicted codes. The correct codes (referred to in the figure as True Positive) and incorrect codes (referred to in the figure as False Positive) are indicated by different gray-scale text in the figure and may be considered to represent a ground truth of the situation. The True Positives in this example are Cat-scratch disease; Acute lymphadenitis of upper limb; and Enlarged lymph nodes, unspecified. The False Positives in this example are Illness, unspecified; Other specified zoonotic bacterial diseases; Zoonotic bacterial disease, unspecified; and Other skin changes.

It can be seen that the meta-refinement step results in the correct ICD code descriptions. This post-processing step may comprise providing the trained model or second trained model, rules and/or other properties of the hierarchical ontology.

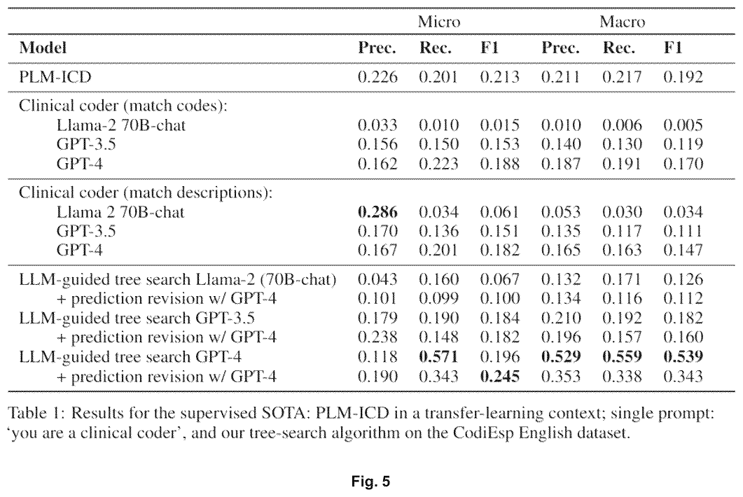

FIG. 5 shows the results of the LLM-guided tree search method compared to a supervised baseline using Pretrained-Language-Model framework (PLM-ICD) and the use of a clinical coder prompt.

The LLM-guided tree search method was validated on the CodiEsp dataset of ICD-coded clinical case documents. CodiEsp is a publicly available dataset which formed the basis of the ‘eHealth CLEF 2020 Multilingual Information Extraction Shared Task’, a competition for automated clinical coding. In this competition, 1000 expert-annotated case notes were released in Spanish, alongside machine-translated English versions. The evaluation was performed on the competition test set, comprising 250 case note documents from 250 unique patients, and covering 1767 distinct ICD-10 codes (2.4% of the ICD-10-CM codeset).

Inspection of the translated documents revealed errors such as failure to translate Spanish terms for drugs. Since translation errors could hamper diagnostic coding performance, the documents were re-translated using GPT-3.5, which reduced errors and yielded modest performance improvements for all models during experimentation. The re-translated dataset is henceforth referred to as the ‘CodiEsp-English’ dataset.

Baseline results are reported from the Pretrained-Language-Model framework (PLM-ICD) which is the state-of-the-art model for the task of ICD coding. It combines BERT as a text encoder model with a per-label attention and per-label binary classifier heads. Due to the limited size of the CodiEsp training dataset (500 case notes), the PLM-ICD used provided model weights learnt on the MIMIC-IV dataset. This represents a realistic transfer learning scenario.

The second baseline is based on the approach of asking the model to act as a clinical coder. This comprises prompting the LLM to assume the role of a clinical coder. A task section of a sample prompt that may be provided to the LLM using a clinical coder approach is “You are a clinical coder, consider the discharge note and assign the appropriate ICD-10 codes, responding with their exact descriptions. Follow the format in the example precisely.”.

Experimentally, it was observed that GPT-4 generates incorrect code-description pairs such as ‘C63.2-Malignant neoplasm of left testis’ where the true description for the ICD code ‘C63.2’ is ‘Malignant neoplasm of scrotum’. We therefore evaluate matching ICD codes in two ways: by the alpha-numeric codes themselves or by their natural language descriptions.

Experimental results are shown in FIG. 5. The evaluation metrics are micro (instances weighted equally) and macro (classes weighted equally) precision, recall and F1 scores. Our baseline, the PLM-ICD model, demonstrated relatively weak performance on the CodiEsp dataset compared to MIMIC-IV, which was used to train it, highlighting the difficulty of transferring between datasets. The ‘Clinical Coder’ method demonstrated modest clinical coding ability, with GPT-4 achieving a micro-F1 score of 0.188; this method has the advantage that it requires only a single prompt. Our tree-search algorithm demonstrated the best predictive ability when combined with the final prediction revision step, achieving a micro-f1 score of 0.245. Closer inspection shows that our prediction revision step (in which codes can be removed but not added) trades recall for better precision.

We note that the ‘clinical coder’ LLM prompt is heavily contingent on the pre-trained knowledge of the particular medical codes and code descriptions in the medical ontology utilized. The tree-search method does not have this requirement, and thus can handle ontology revisions such as ICD-11 (introduced in January 2022) or new codes such as the COVID-19 code UO7.1 (introduced in February 2020).

In certain embodiments using a smaller LLaMa model, the output received was found to have poor accuracy (such as all codes predicted as ‘no’) and was not parseable. The results of a clinical coding operation on a smaller LM such as LLaMa may be improved by making the in-context learning example more structured. For instance, ICD codes and predictions (e.g. a yes/no output in this example) may be included in the prompt as an html table of results. However, this means that a large part of the input prompt will need to be dedicated to showing the output format, and a large part of the generated output will be boilerplate code, such as e.g. html tags. State machines have been used to force particular tokens at give positions with an LLM generating the remaining tokens in order to generate a structures output. In this way, the LLM can be made to deterministically follow a desired output schema, such as a JSON structure. Using deterministic structured output for responses would make the methods presented in this disclosure more robust, even when smaller LLMs are used, as well as further increasing their efficiency. There are various potential benefits to using such a technique, for example:
1) The output of the method may be guaranteed to adhere to the desired output schema exactly.
2) Generation of output data may be more efficient with this technique. A majority, approximately 75% of the output generation required will be dispensed with since most tokens in the output are fixed by design. The model then only needs to generate binary (‘yes’/‘no’) predictions for each code along with any relevant evidence for its decision, rather than the entire output structure. This would yield a faster prediction wherein the speed improvement would be proportionate to the reduction in generation. For instance, in one example, all parts of the output 58 could be fixed except the single (yes/no) token per line, even forcing the model to elide its explanation of its prediction if desired.
3) There may be more space in the prompt to provide details about the actual prediction task, which may yield some predictive improvements.

Embodiments have been described that provide clinical coding, for example based on ICD codes. Embodiments are not limited to such applications, and other embodiments can be applied not only for ICD, but also or instead for a tag and/or annotation assignment to medical text, if such the tag and/or annotation has a tree/node structure with definitions at each node. For example, embodiments could be used to summarise and/or tag and/or catalog any suitable medical text using terms and/or tags from a hierarchical ontology.

In certain embodiments, a method for performing clinical coding from clinical text documents is described, the method comprising assessing a hierarchical code ontology, with associated text descriptions for each code using a pre-trained language model and a target clinical document to be coded, using a coding agent to perform recursive tree search of the hierarchical code ontology and using the pre-trained language model to determine which paths and nodes it follows or visits through the tree. A prompt structure may be used for a language model that contains the target clinical document, an in-context training example, and an information retrieval task relating to the child codes of the node being visited. The method may be followed by a meta-refinement or post-processing step in which a language model is prompted to eliminate false positive codes from the list of assigned clinical codes. The prompt may further comprise relevant ICD rules and conventions and a deterministic structured output mechanism for LLM responses may be employed.

According to various embodiments there is provided a data processing apparatus for obtaining an output corresponding to a medical text input, the apparatus comprising processing circuitry configured to:
provide a medical text input, or data derived from the medical text input, to a trained model;
provide instructions to the trained model, for example to repeatedly assess layers of a hierarchical ontology that comprises a plurality of nodes at each of a plurality of layers, for instance thereby to determine path(s) through the layers via node(s) in successive layers that are connected according to the hierarchical ontology and that match the medical text input. The processing apparatus may be configured to select at least one node from the node(s) at the end or other point(s) of the determined path(s). The processing apparatus may be configured to select no nodes if, for example, there are no suitable matches of the medical text input to nodes. The processing apparatus may be configured to output text, or other data, associated with the selected at least one node.

It is possible for no nodes to be selected, if for example the medical text input contains no information which merits coding. Also, in some embodiments, some paths may terminate before reaching a leaf node e.g. if a concept retrieved from the text has no associated concept in the ontology (or the model believes it does not), or the concept is located elsewhere in the ontology e.g. the concept “lung cancer” might lead to exploration of branches relating both to “respiratory disease” and “neoplasms”.

The assessment by the trained model may comprise performing a recursive tree search of the hierarchical ontology to determine the path(s) through the ontology.

The selection of path(s) may be such that only a sub-set of nodes of the hierarchical ontology are subject to assessment by the trained model in order to obtain the selected nodes, and/or
the selection of path(s) may be such as to provide sparse exploration of the hierarchical ontology in order to obtain the selected nodes.

The providing of instructions and/or the providing of the medical text input may comprise providing one or more prompts to the model, wherein the prompt(s) comprise medical text input or derived data, and/or an in-context training example, and/or an information retrieval task relating to one or more of the nodes.

The providing of instructions may comprise, for each node of the path(s), providing text associated with each of the child nodes of said node and instructing the trained model to determine if any of the associated text is included in, or otherwise matches, the medical text input.

The processing circuitry may be configured to perform a post-processing step to eliminate one or more of the selected nodes, or their associated text, for example thereby to eliminate one or more false positives.

The processing circuitry may be configured to instruct the trained model, or a further trained model, to perform the post-processing step.

The instructing of the trained model, or the further trained model, to perform the post-processing step may comprise providing at least some rules or other properties of the hierarchical ontology to the trained model or the further trained model, for example thereby to assist in eliminating false positives.

The assessment by the trained model may comprise using the trained model as a discriminator function in a multi-label decision tree process performed on the hierarchical ontology.

The discriminator function may act on respective text associated with each node of the hierarchical ontology to select relevant nodes thereby to obtain the path(s) through the layers via node(s) in successive layers.

The trained model may be instructed to select a node as relevant if there is a match between at least some of the text associated with the node and at least some of the text of the medical text input.

The model may be trained on training data that includes text that is different from or additional to text of the hierarchical ontology.

The model may be trained on a training data set that does not include training data for at least some nodes of the hierarchical ontology and/or text associated with those nodes, and/or wherein the trained model may be such as to perform a zero shot process in respect of at least some of the nodes.

The model may comprise a large language model (LLM) or other language model, and/or the providing of instructions to the model may comprise sending instructions via an API to provide desired input to the model. The LLM may, for example, have billions of parameters.

The model may comprise at least one of GPT-2, GPT-3.5, GPT-4, PaLM, LLaMa, BLOOM, Ernie, T5, Claude or Claude 2 or any suitable derivatives or developments thereof.

The hierarchical ontology may comprise the International Classification of Disease (ICD), SNOMED CT, Radlex or other diagnostic code ontology.

The medical text input may comprise at least one of medical notes for a patient or other subject, results of a diagnostic or other procedure, test or scan results or text associated with such results.

The output may comprise a text or code input for use in at least one of billing, audit, resource management, epidemiological study, measurement of treatment effectiveness, insurance processing, or enhancement of medical records.

The outputting of the text, or other data, associated with the selected at least one node may comprise generating the text, or other data, using the or a trained model.

According to various embodiments there is provided a data processing method comprising:
providing a medical text input, or data derived from the medical text input, to a trained model;
providing instructions to the trained model to repeatedly assess layers of a hierarchical ontology that comprises a plurality of nodes at each of a plurality of layers, thereby to determine path(s) through the layers via node(s) in successive layers that are connected according to the hierarchical ontology and that match the medical text input;
selecting at least one node from the node(s) at the end or other point(s) of the determined path(s); and
outputting text, or other data, associated with the selected at least one node. The method may comprise select no nodes if, for example, there are no suitable matches of the medical text input to nodes.

In another aspect, there is provided a computer program product comprising computer-readable instructions that are executable to perform a method as claimed or described herein.

In a further aspect, which may be provided independently, there is provided a method and/or apparatus for performing clinical coding from clinical text documents, comprising a hierarchical code ontology, with associated text descriptions for each code, a pre-trained language model, a target clinical document to be coded, a coding agent performing recursive tree search of the hierarchical code ontology, using the pre-trained language model to determine which paths (nodes) it follows (visits) through the tree, and a prompt structure for a language model that contains at least one of the target clinical document, and/or an in-context training example, and/or an information retrieval task relating to the child codes of the node being visited.

There may be provided a meta-refinement post-processing step in which a language model is prompted to eliminate false positive codes from the list of assigned codes.

There may be provided a post-processing step in which ICD rules and conventions are provided to the model as part of the prompt instruction.

Features in one aspect or embodiment may be combined with features in any other aspect or embodiment in any appropriate combination. For example, apparatus features may be provided as method features and vice versa.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments are described, these embodiments have been presented by way of example only, and are not intended to limit the scope of the invention. Indeed, the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A data processing apparatus for obtaining an output corresponding to a medical text input, the apparatus comprising processing circuitry configured to:
provide a medical text input, or data derived from the medical text input, to a trained model;
provide instructions to the trained model to repeatedly assess layers of a hierarchical ontology that comprises a plurality of nodes at each of a plurality of layers, thereby to determine path(s) through the layers via node(s) in successive layers that are connected according to the hierarchical ontology and that match the medical text input;
select at least one node from the node(s) at the end or other point(s) of the determined path(s), or select no nodes if there are no suitable matches of the medical text input to nodes; and
output text, or other data, associated with the selected at least one node.

2. An apparatus according to claim 1, wherein the assessment by the trained model comprises performing a recursive tree search of the hierarchical ontology to determine the path(s) through the ontology.

3. An apparatus according to claim 1, wherein the selection of path(s) is such that only a sub-set of nodes of the hierarchical ontology are subject to assessment by the trained model in order to obtain the selected nodes, and/or
wherein the selection of path(s) is such as to provide only sparse exploration of the hierarchical ontology in order to obtain the selected nodes.

4. An apparatus according to claim 1, wherein the providing of instructions and/or the providing of the medical text input comprises providing one or more prompts to the model, wherein the prompt(s) comprise medical text input or derived data, and/or an in-context training example, and/or an information retrieval task relating to one or more of the nodes.

5. An apparatus according to claim 1, wherein the providing of instruction comprises, for each node of the path(s) instructing the trained model to determine if any text associated with child nodes of said node is included in, or otherwise matches, the medical text input.

6. An apparatus according to claim 1, wherein the processing circuitry is configured to perform a post-processing step to eliminate one or more of the selected nodes, or their associated text.

7. An apparatus according to claim 6, wherein the processing circuitry is configured to instruct the trained model, or a further trained model, to perform the post-processing step.

8. An apparatus according to claim 7, wherein the instructing of the trained model, or the further trained model, to perform the post-processing step comprises providing at least some rules or other properties of the hierarchical ontology to the trained model or the further trained model thereby to assist in eliminating false positives.

9. An apparatus according to claim 1, wherein the assessment by the trained model comprises using the trained model as a discriminator function in a multi-label decision tree process performed on the hierarchical ontology.

10. An apparatus according to claim 9, wherein the discriminator function acts on text associated with nodes of the hierarchical ontology to select relevant nodes thereby to obtain the path(s) through the layers via node(s) in successive layers.

11. An apparatus according to claim 10, wherein the trained model is instructed to select a node as relevant if there is a match between at least some of the text associated with the node and at least some of the text of the medical text input.

12. An apparatus according to claim 1, wherein the model is trained on training data that includes text that is different from or additional to text of the hierarchical ontology.

13. An apparatus according to claim 1, wherein the model is trained on a training data set that does not include training data for at least some nodes of the hierarchical ontology and/or text associated with those nodes, and/or wherein the trained model is such as to perform a zero shot process in respect of at least some of the nodes.

14. An apparatus according to claim 1, wherein the model comprises a large language model (LLM) or other language model, and/or where the providing of instructions to the model comprises sending instructions via an API to provide desired input to the model.

15. An apparatus according to claim 14, wherein the model comprises at least one of GPT-2, GPT-3.5, GPT-4, PaLM, LLaMa, BLOOM, Ernie, T5, Claude or Claude 2, or any suitable derivatives or developments thereof.

16. An apparatus according to claim 1, wherein the hierarchical ontology comprises the International Classification of Disease (ICD), SNOMED CT, Radlex or other diagnostic code ontology.

17. An apparatus according to claim 1, wherein the medical text input comprises at least one of medical notes for a patient or other subject, results of a diagnostic or other procedure, test or scan results or text associated with such results.

18. An apparatus according to claim 1, wherein the output comprises a text or code input for use in at least one of billing, audit, resource management, epidemiological study, measurement of treatment effectiveness, insurance processing, or enhancement of medical records.

19. An apparatus according to claim 1, wherein the outputting of the text, or other data, associated with the selected at least one node comprises generating the text, or other data, using the or a trained model.

20. A data processing method comprising:
providing a medical text input, or data derived from the medical text input, to a trained model;
providing instructions to the trained model to repeatedly assess layers of a hierarchical ontology that comprises a plurality of nodes at each of a plurality of layers, thereby to determine path(s) through the layers via node(s) in successive layers that are connected according to the hierarchical ontology and that match the medical text input;
selecting at least one node from the node(s) at the end or other point(s) of the determined path(s), or selecting no nodes if there are no suitable matches of the medical text input to nodes; and
outputting text, or other data, associated with the selected at least one node.
